(19) United States
(12) Patent Application Publication
(10) Pub. No.: US 2016/0354519 A1
(43) Pub. Date: Dec. 8, 2016

(54) METHOD FOR PREPARING BIOLOGICAL TISSUE

(71) Applicant: BIOTRONIK AG, Buelach (CH)

(72) Inventors: Alexander Rzany, Nuernberg (DE); Wilhelm Erdbruegger, Konstanz (DE)

(21) Appl. No.: 15/170,528

(22) Filed: Jun. 1, 2016

(30) Foreign Application Priority Data
Jun. 8, 2015 (DE) 10 2015 108 952.1

(51) Int. Cl. A61L 27/36; A61L 27/50; A61L 27/24

(52) U.S. Cl. CPC A61L 27/3687; A61L 27/3625; A61L 27/24; A61L 27/507

(57) ABSTRACT

A method for preparing tissue for medical applications, in particular for preparing tissue for use for an artificial heart valve using an α-galactosidase.

### CROSS REFERENCE TO RELATED APPLICATIONS

This application claims benefit of priority to German patent application no DE 10 2015 108 952.1 filed Jun. 8, 2015, the content of which is herein incorporated by reference in its entirety.

### TECHNICAL FIELD

The present invention relates to a method for preparing tissue for medical applications, in particular for preparing tissue for use for an artificial heart valve.

### BACKGROUND

The present invention is indeed particularly suitable for preparing tissue for use for an artificial heart valve, but is not limited to this application. The present invention can also be applied for the preparation of blood vessels, bone, cartilage, ligaments, corneas or the like.

Mechanical heart valve prostheses and the associated accompanying medications can involve significant limitations for the patient. Patients who have a mechanical heart valve must undergo anticoagulation treatment for the rest of their life and therefore are at increased risk permanently of thromboembolic complications and bleeding.

In order to avoid these disadvantages and difficulties, biological heart valve prostheses based on human tissue (as allograft or homograft) or animal tissue (as xenograft) have been developed. In the development of biological heart valve prostheses, in particular in prostheses having valve leaflets made of biological material of animal origin (what are known as xenografts), it is possible to dispense with long-term anticoagulation. However, biological heart valve prostheses tend towards calcification, wherein the calcification can lead to a loss of function of the prosthesis. It has been demonstrated that, in spite of extreme care and attention when preparing the biological material, even small residues of proteins or cells that have not completely denatured following the implantation can lead to a biological response. Premature calcifications of the biological valve material are observed as a result. The biological heart valve prosthesis fails and may have to be replaced. Besides the calcification, the continuous mechanical loading of the prostheses also constitutes a problem, and therefore biological heart valve prostheses usually have a shorter service life than mechanical heart valve prostheses.

The heart valve formed of biological tissue is usually secured in a main body (for example a rigid plastic framework or a self-expanding stent), which is then implanted at the position of the natural valve. The present invention describes a method for preparing such tissue for use in a heart valve prosthesis for implantation at the site of a natural heart valve.

The tissue of origin must be subjected before implantation to a procedure having a number of steps in order to be able to be implanted as prepared tissue. In so doing, the tissue is modified, to the greatest extent possible, such that the tissue is not recognized by the body as foreign tissue, is not calcified, and has the longest life span possible. Such a method for preparing tissue substantially comprises at least two main steps having a plurality of intermediate rinsing processes, wherein the tissue of origin has to be thoroughly cleaned and prepared prior to the actual treatment steps.

The first essential preparation step is what is known as the decellularization of the tissue. In this step, cell membranes, intracellular proteins, cell nuclei, and other cellular components are removed as completely as possible from the tissue in order to obtain the purest extracellular matrix possible. Any cells and cellular components remaining in the tissue would be potent starting points, in particular, for an unwanted calcification of the biological implant material. The decellularization, as a washing step, should be performed in a manner that is so gentle that the structure and the collagen fibers in the extracellular matrix remain as unaffected as possible while ensuring that all cells contained therein are thoroughly removed from the tissue. Cleaned, but otherwise unaffected, intact collagen fibers are preferred, since these ensure the mechanical stability of the further-processed tissue.

The second essential preparation step is cross-linking the tissue, in particular the collagen fibers. After decellularization, preferably all cellular components have been removed from the tissue and the biological material nearly exclusively consists of the extracellular matrix. In the case of pericardial tissue, the extracellular matrix is formed primarily of collagen fibers. In order to obtain biological material having the most optimal mechanical properties possible and to prevent rejection reactions by the receiving body, the collagen fibers are cross-linked by means of a suitable cross-linking agent via the incorporation of chemical bonds. The cross-linking agent binds to the amino groups of the collagen fibers and forms chemically stable bonds between collagen fibers. A biological material having long-term stability is thereby obtained from the three-dimensionally arranged collagen fibers, wherein this biological material is no longer recognized as foreign biological material. The stability and strainability of the tissue is markedly increased by means of the three-dimensional cross-linking or linking of the individual collagen fibers via the cross-linking agent. This is decisive, in particular, in the case of use as tissue of a heart valve, where the tissue must open and close, at one-second intervals, as a valve.

The above-mentioned calcifications can occur, however, in spite of carefully performed decellularization and can originate, inter alia, in antibodies directed against galactose-α-1,3-galactose-β-1,4-N-acetylglucosamine epitopes (α-gal epitopes on the surface of the implanted tissue). Here, α-gal epitopes can lead to severe immune responses that encourage calcification. The concentration of α-gal epitopes on the surface could be reduced in principle by harsh decellularization conditions. However, this would have a significantly negative influence on the mechanical properties of the valve material. In order to minimize calcifications and provide tissue having significantly improved mechanical properties, it would therefore be desirable to provide tissue that has been subjected to gentle decellularization and with which α-gal epitopes on the surface of the tissue have been fully removed where possible.

### SUMMARY

In light of the above, methods for preparing tissue for use in medical applications are provided including a step of treating the tissue with an α-galactosidase, which may be performed after a decellularization step and before a crosslinking step, and a tissue obtained from the methods.

In particular, a method for preparing biological tissue is proposed, which method makes it possible to remove cellular components and α-gal epitopes thoroughly, but gently from the to tissue in such a way that a subsequent cross-linking produces a mechanically stable and durable tissue, which in particular is suitable for use as tissue of an artificial heart valve and has a significantly reduced tendency towards calcification. This is provided by the method proposed herein.

In light of the above, a method is provided for preparing tissue for medical applications, in particular tissue for use for an artificial heart valve. A method is provided that includes a step of decellularizing the tissue by means of a detergent. As a result of the decellularization, the cellular components are removed from the tissue and the biological material consists almost exclusively of the extracellular matrix. A step for treating the tissue with at least one α-galactosidase is also proposed. By means of the treatment with at least one α-galactosidase, α-gal epitopes on the surface of the tissue can be removed, and the risk of a subsequent calcification can be significantly reduced or even minimized. In a preferred embodiment the tissue is treated with one α-galactosidase. However, it is likewise possible to use a combination of α-galactosidases. This means that the α-galactosidases used in combination have a different structure and/or origin; i.e. the α-galactosidases have been produced in a different living organism and/or have a different structure.

It is also provided that the methods can include a step of cross-linking the extracellular matrix of the tissue by means of a suitable cross-linking agent. In a preferred embodiment the extracellular matrix is present in the form of collagen fibers. The cross-linking agent binds to the amino groups of the collagen fibers and forms chemically stable bonds between the collagen fibers. A biological material that is stable in the long-term and that additionally is no longer recognized as biological foreign material is thus created from the three-dimensionally arranged collagen fibers. Furthermore, the tissue is mechanically more stable as a result of the cross-linking.

The invention provides that α-galactosidases, also α-D-galactoside galactohydrolases, E.C. 3.2.1.22, are enzymes that are able to catalyze the hydrolysis of galactosyl residues of the non-reducing ends of a multiplicity of oligosaccharides and polysaccharides and also of galactolipids and glycoproteins. With regard to tissue, α-galactosidases can be used to remove α-1,3-galactosyl residues on and in the tissue. It has been found here that α-gal epitopes can be effectively removed from the surface of the tissue by treatment with α-galactosidases, whereby immune responses and calcifications can be reduced.

It is also provided that α-galactosidases can vary significantly in terms of their purpose, structure and effect, depending on their origin. This is strongly associated with the fact that a multiplicity of organisms produce α-galactosidases, such as archaea, bacteria, fungi, plants or animals. A possible grouping of α-galactosidases may lie in the purpose of the organism in question. Here, a grouping may be given for example by the pH-dependency of the enzyme activity. The inventors have found that not all α-galactosidases are equally suitable for the treatment of tissue for use in heart valves. Rather, the inventors have surprisingly found that certain α-galactosidases are suitable in particular for use in the method proposed herein.

In a preferred embodiment the use of alkaline α-galactosidases is provided for the methods herein. Alkaline α-galactosidases are characterized in that they have a high or their highest enzymatic activity in alkaline medium and also have a high substrate specificity. The use of alkaline α-galactosidases is advantageous, since it is thus made possible to also use DNases and RNases parallel to the alkaline α-galactosidases. DNases and RNases are used for the removal of residual ribonucleic acids from the tissue, which may also contribute to a calcium binding. By combination of α-galactosidases with DNases and/or RNases, an even more improved protection against calcification can thus be achieved, in particular in a pH range from 7.1 to 8.0, more preferably in a pH range from 7.2 to 7.8, and most preferably that demonstrate the highest specific enzyme activity in a pH range from 7.3 to 7.6. Preferred alkaline α-galactosidases originate from Arabidopsis thaliana, Cucumis melo, Cucumis sativus, Oryza sativa, for example the Japonica group, Pisum sativum, Solanum Lycopersicum, Tetragonia tetragonioides and Zea mays.

In a further preferred embodiment α-galactosidases from the GH-36 family are used in the methods. It has been found that representatives of the GH-36 family can remove α-gal epitopes on tissue highly efficiently. The inventors have surprisingly found that α-galactosidases from the GH-36 family can remove α-gal epitopes on tissue more quickly and at lower concentrations than representatives from other GH families.

In a further preferred embodiment α-galactosidases from the GH-36 family, sub-group II, are used in the methods. Representatives of this sub-group have proven to be particularly suitable for efficiently removing α-gal epitopes on tissue. Preferred α-galactosidases from the GH-36 family, sub-group II, are based on the following sources, selected from the group comprising or consisting of Oryza sativa of the Japonica group, Cucumis melo, Bifidobacterium breve C50, Sulfolobus solfactarius and Sulfolobus tokodaii. In a particularly preferred embodiment of the method proposed herein, the α-galactosidase originates from Cucumis melo.

It has been found in particular that α-galactosidases of Cucumis melo are able to remove α-gal epitopes on tissue more specifically than, for example, α-galactosidases of green coffee bean (GCB) or the acidic variant of Aspergillus niger.

As presented above, the proposed method can have additional steps besides the treatment of tissue with α-galactosidases. This is advantageous in particular in respect of the acquisition of a tissue that has both excellent mechanical properties and low to no tendency towards calcification.

In accordance with the methods a tissue is subjected to decellularization with a detergent, wherein lipopeptides are preferably used as detergent. In accordance with the present invention, peptides containing β-hydroxy fatty acid or β-amino hydroxy fatty acid, i.e. lipopeptides, are not used for conditioning, but as detergent for decellularization. It has been found that the lipopeptides provide excellent results in the decellularization of tissue. The tissue is freed of cellular components much more gently. The structure of the extracellular matrix is very well retained with the use of an above-mentioned detergent for decellularization, and forms an improved starting basis for a treatment according to the invention with α-galactosidases as described herein. A suitable detergent for the present method thus contains at least one lipopeptide having amphiphilic properties, consisting essentially of or consisting of a hydrophilic structure and a hydrophobic side chain. A tissue that has much better mechanical properties compared to the prior art and is therefore suitable in particular for use in a heart valve prosthesis can thus be attained by the preferably subsequent cross-linking step. Together with a treatment step with α-galactosidases as described herein, tissue can be provided that has much better mechanical properties and tends towards calcification and immune response to a minimal extent. If the tissue is introduced into an implant, such as an artificial heart valve, these are characterized by good mechanical stability on the whole and a long service life.

The detergent for decellularization particularly preferably contains a cyclic lipoheptapeptide, in particular surfactin. In this preferred embodiment of the invention a surfactin-containing detergent is used for decellularization. In particular, the detergent contains surfactin having a cyclical structure, as presented below:

(CH₃)₂—CH—(CH₂)→CH—CH₂—CO→L-Glu→L-Leu→D-Leu→L-Val→L-Asp→D-Leu→L-Leu→O

Here, n=8-12, preferably 9, and Glu, Leu, Val, Asp stand for the amino acids, glutamic acid, leucine, valine and aspartic acid.

Lipopeptides such as daptomycin, caspofungin, arthrofactin, echinocandine, iturine, syringomycine, syringopeptide and/or polymyxin are also advantageous. The detergent expediently contains at least one lipopeptide selected from the following list: surfactin, daptomycin, caspofungin, arthrofactin or the group of echinocandine, iturine, syringomycine, syringopeptide, polymyxin.

The detergent particularly preferably consists of a buffer solution, particularly preferably a phosphate buffer solution, expediently at pH 7.4, which contains the lipopeptide, particularly surfactin, at a concentration from 100 mg/l to 2000 mg/l, preferably 500 mg/l to 700 mg/l, particularly preferably 600 mg/l. Here, the use of Dulbecco's phosphate-buffered saline solution (DPBS) without calcium and magnesium as carrier solution for the detergent surfactin is particularly advantageous. Other biological buffer solutions, such as tris(hydroxymethyl) aminomethane (TRIS)-buffered or 2-(4-(2-hydroxyethyl)-1-piperazinyl) ethanesulfonic acid (HEPES)-buffered solutions are likewise expedient.

The tissue is advantageously rinsed during the method proposed herein before and particularly preferably after the decellularization at least once, preferably more times, with a suitable solvent, in particular a buffered saline solution and/or an alcohol solution.

The methods herein may additionally include a cross-linking step. The cross-linking agent preferably contains glutaraldehyde. In alternative embodiments of the invention the cross-linking agent contains carbodiimide, aldehydes, such as formaldehyde, glutaraldehyde acetals, acyl azides, cyanimide, genepin, tannin, pentagalloyl glucose, phytate, proanthocyanidin, reuterin and/or epoxy compounds.

Here, buffered sodium chloride solutions and/or an ethanol solution are particularly advantageous.

In principle, all types of tissue from mammals, including humans, can be used for the proposed method. Non-human tissue is preferred. In particular, tissues that can be used as valve material in a heart valve are suitable. Here, pericardial tissue and heart valves, but also skin, connective tissue, tendons, peritoneal tissue, dura mater, tela submucosa, in particular of the gastrointestinal tract, or pleura are preferred. In the case of heart valves all valves can be used, i.e. aortic, pulmonary, mitral and tricuspid valves. Furthermore, pericardial tissue from pig, sheep, goat, horse, crocodile, kangaroo, ostrich and cattle are preferred.

The use of α-galactosidases as described herein for the treatment of biological tissue, in particular of biological tissue for heart valve prostheses, is also proposed. In particular, the use of alkaline α-galactosidases is proposed. Furthermore, the use of α-galactosidases of the GH-36 family, preferably sub-group II, and more preferably of Cucumis melo is proposed.

The use of an α-galactosidase as described herein for the treatment of biological tissue is particularly advantageous in conjunction with the use of a solution containing at least one lipopeptide, in particular surfactin, daptomycin, caspofungin, arthrofactin, an to echinocandin, an iturin, a syringomycin, a syringopeptide and/or a polymyxin as detergent for decellularization of biological tissue. Such a combination of treatment steps provides tissue in which cellular material and α-gal epitopes have been removed very gently and comprehensively.

The present invention in particular provides a method for preparing biological tissue that ensures a thorough and reliable decellularization which at the same time is carried out in a manner that is gentle on the tissue, such that the mechanical properties of the tissue after decellularization, treatment with α-galactosidase and cross-linking are much better compared with the prior art.

The method according to the invention for preparing biological tissue, in particular for preparing biological tissue for use in a heart valve prosthesis, additionally minimizes the risk of calcification of the tissue (and therefore of the prosthesis) in clinical use by means of the treatment with α-galactosidases as described herein. Due to the treatment according to the invention with at least one α-galactosidase, in particular in conjunction with a decellularization with a detergent as described herein, the properties of the tissue are significantly positively influenced. A tissue prepared in accordance with the method according to the invention demonstrates a much better mechanical load-bearing capability and significantly reduced tendency to trigger an immune response and to calcify. Furthermore, tissue that has both been treated with α-galactosidase and decellularized with a detergent described herein, preferably surfactin, once again has improved surface properties.

The invention will be explained in greater detail hereinafter on the basis of the exemplary embodiments in the drawings. Here, the influence of different α-galactosidases on the concentration of α-gal epitopes on the surface of the tissue will be examined.

In one exemplary embodiment of the invention a biological tissue is obtained from porcine pericardial tissue by mechanical removal of adhering foreign tissue and subsequent rinsing in isotonic saline solution (company Fresenius-Kabi) for 2 hours at 4 C. This tissue was subjected to a decellularization with a detergent consisting of a DPBS solution (Dulbecco's phosphate buffered saline) without calcium/magnesium (company Lonza; DPBS w/o Ca++/Mg++; product no. 17-512) and surfactin (company Sigma-Aldrich, Surfactin from Bacillus subtilis, product no. F3523) in a concentration of 600 mg/l, for 20 hours at 37° C.

The decellularized tissue was then mixed with α-galactosidases of green coffee beans and of sugar melon (Cucumis melo) at a concentration of 5 units per ml, 1 unit per ml, and 0.1 units per ml for 24 hours. A “unit” is understood to mean the amount of enzyme that can hydrolyze 1.0 μmol of p-nitrophenyl-α-D-galactoside into p-nitrophenol and D-galactose per min at pH 6.5 and 25° C. in DPBS.

In order to conclude how effectively α-gal epitopes have been removed from the tissue, the absorption of specific IgM antibodies (M86) of the treated tissue is measured. The fewer IgM antibodies are absorbed into the tissue, the fewer α-gal epitopes remain in the tissue. Furthermore, a comparison is made with non-decellularized tissue, although this has been treated with α-galactosidases in the above-mentioned way.

### BRIEF DESCRIPTION OF THE DRAWINGS

FIG. 1 is a graph depicting the absorption of M86 antibodies on native tissue with and without decellularization and treatment with α-galactosidase.

FIG. 2 is a graph depicting the absorption of M86 antibodies on native tissue with and without decellularization and treatment with an α-galactosidase of Aspergillus niger.

FIG. 3 is a graph depicting the measured protein from porcine pericardium before and after decellularization with surfactin.

FIG. 4 is a graph depicting the measured DNA from porcine pericardium before and after decellularization with surfactin.

FIG. 5 is a graph depicting a comparison of shrinkage temperature of decellularized tissue after treatment with the three detergents surfactin, deoxycholic acid (DCA) and sodium dodecyl sulfate (SDS) compared with the shrinkage temperature of the native tissue.

FIGS. 6a-6d are images taken from electron microscope of native tissue (FIG. 6a) and of the tissue following decellularization with surfactin, DCA, or SDS (FIGS. 6b, 6c, 6d).

### DETAILED DESCRIPTION

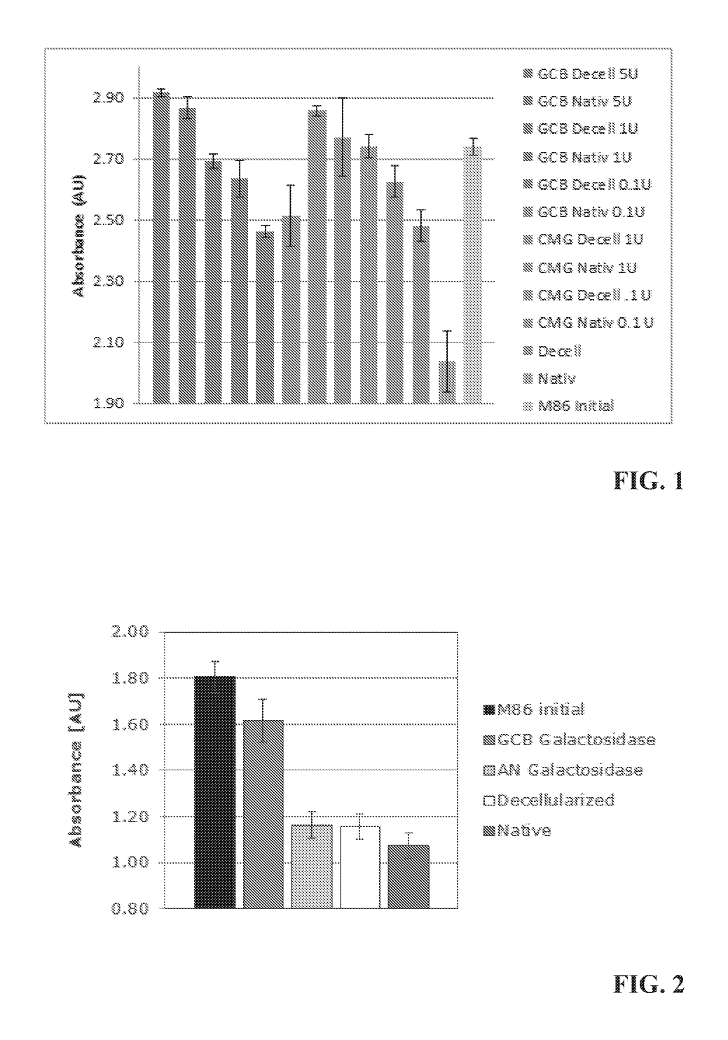

FIG. 1 shows the absorption of M86 antibodies on the treated tissue. In the graph, two types of tissue are compared: native tissue, which has not been decellularized, and decellularized tissue. Comparison values are contained on the right-hand side of the graph: M86 initial, Nativ and Decell give the absorption values for tissue that has not been treated with α-galactosidase. Here, the native tissue demonstrates the highest value of α-gal epitopes. M86 initial specifies the absorption at which no absorption of the antibodies has taken place. This value constitutes the limit value for tissue on which α-gal epitopes are no longer present. From the comparison of M86 initial, Nativ and Decell, it can be seen that the decellularization already removes a significant quantity of α-gal epitopes (comparison of Decell and Nativ). However, it is also clear that a significant quantity of α-gal epitopes remain on the tissue (comparison of Decell and M86 initial).

The further absorption data shows the influence of the treatment with α-galactosidases on the concentration of α-gal epitopes on the surface of the tissue. The α-galactosidases of green coffee bean (GCB, Sigma Aldrich) at a concentration of 1 unit per ml could not remove all α-gal epitopes (comparison of M86 initial and GCB*5 U). However, due to the use of 1 U of the α-galactosidase of green coffee bean, the concentration of α-gal epitopes on the surface is considerably reduced (comparison of Decell/Nativ and GCB*1 U). If the high concentration of 5 Units per ml of the α-galactosidase of green coffee bean is used, practically all α-gal epitopes on the surface of the tissue can be removed (comparison of M86 initial and GCB*5 U). The extraordinary suitability of the α-galactosidase of Cucumis melo (CMG, Cucumis melo galactosidase) will be explained hereinafter on the basis of FIG. 1. If the comparatively low concentration of 1 unit per ml is used, all α-gal epitopes on the surface of the tissue can be removed (comparison of M86 initial with CMG*1 U). It has also been found that in the case of decellularized tissue just 1/10 of a unit is sufficient to remove practically all α-gal epitopes on the surface of the tissue (comparison of M86 initial with CMG Decell 0.1 U). In the case of native tissue approximately all α-gal epitopes on the surface of the tissue are removed at this extremely low concentration (comparison of M86 initial with CMG Native 0.1 U). It has thus been found that α-galactosidases of Cucumis melo can remove α-gal epitopes on the surface of the tissue in a highly efficient manner, and moreover much better than α-galactosidases of green coffee bean.

FIG. 2 shows, in addition to the above data, the relative performance of an α-galactosidase of Aspergillus niger. Again, the comparison values of M86 initial, Native and Decellularized are shown, wherein M86 initial again describes the value at which it is assumed that α-gal epitopes are no longer present on the surface of the tissue, whereas Native and Decellularized specify the values of tissue that has not been treated with α-galactosidase. Native tissue at a concentration of 5 units/ml forms the basis. It can be seen, as already clear from FIG. 1, that the α-galactosidase of green coffee bean (GCB) is able to remove α-gal epitope on the surface of the tissue. By comparison, however, it can be seen that the acidic α-galactosidase of Aspergillus niger (AN) is hardly able at this concentration to remove α-gal epitopes on the surface of the tissue.

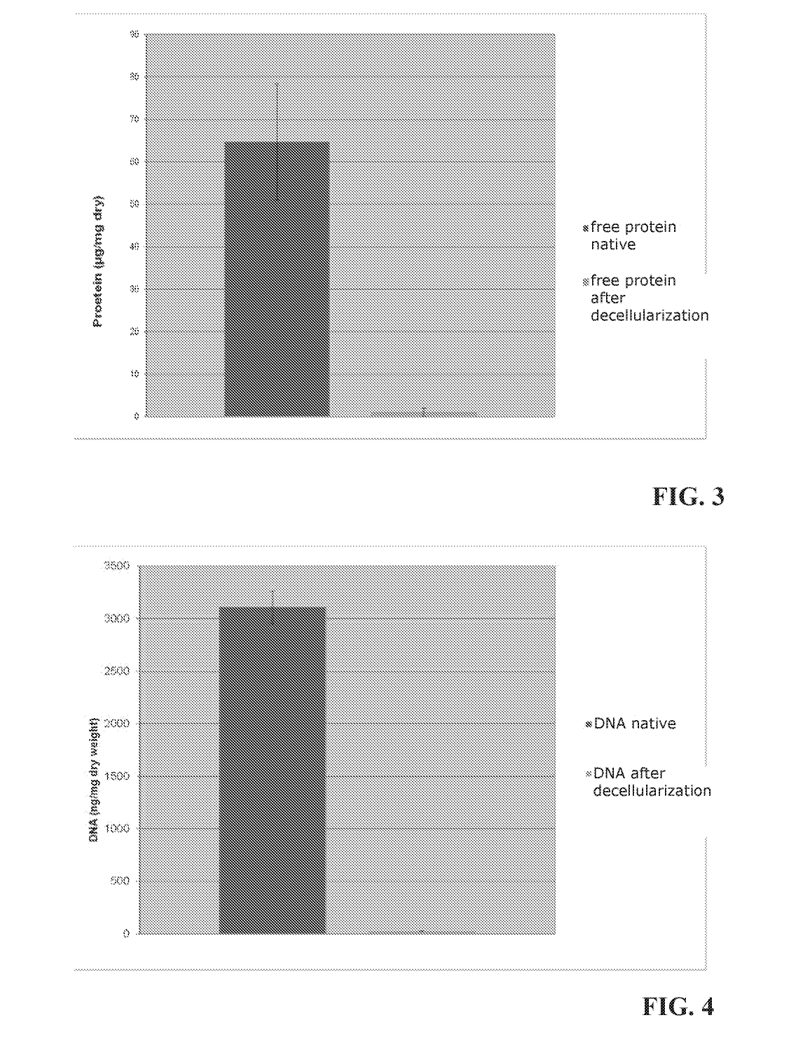

FIG. 3 shows the influence of decellularization with surfactin at a concentration of 0.06% in DPBS without Ca²⁺/Mg²⁺ within 20 hours on the protein content of porcine pericardium. As shown in FIG. 3, the decellularization leads to a significant reduction of proteins.

In FIG. 4, similarly to FIG. 3, the influence of the decellularization described under FIG. 3 on the DNA content of porcine pericardium is illustrated. The decellularization leads to a significant decrease of DNA molecules.

FIG. 5 shows on the ordinate (enlarged scale, no zero point shown) the shrinkage temperature of the decellularized tissue after treatment with the three detergents surfactin, deoxycholic acid (DCA) and sodium dodecyl sulfate (SDS) compared with the shrinkage temperature of the native tissue.

On account of the dominating proportion of collagen in the extracellular matrix of pericardial tissue, the shrinkage temperature is the temperature at which the protein collagen thermally denatures, i.e. changes irreversibly in terms of its three-dimensional structure. As a result of the structural change to the collagen molecules, massive irreversible structural changes are produced in the tissue, which is clearly visibly smaller when the shrinkage temperature is reached.

The shrinkage temperature is determined by way of experiment by means of differential scanning calorimetry (DSC). In this method the temperature of the sample to be measured increases linearly with time, and the flow of heat into and from the sample is measured with respect to a reference sample. If thermodynamic processes occur in the sample, for example the irreversible structural change of the collagen, a noticeable peak occurs in the measured thermogram at the shrinkage temperature. The level of the shrinkage temperature is a direct indicator for the stability of the three-dimensional structure of the collagen molecules. A minimal change compared with the state in the native tissue is therefore direct proof at molecular level of the much more gentle decellularization by surfactin.

As is clearly shown in FIG. 5, the shrinkage temperature of the pericardial tissue following decellularization with a lipopeptide described herein is practically identical to the shrinkage temperature of the untreated native pericardial tissue. The decellularization according to both exemplary embodiments with DCA and SDS by contrast lead to a shrinkage temperature significantly reduced by 3° C. and 5° C. respectively, and therefore a significantly compromised tissue structure. The mechanical properties of the native biological tissue and of the tissue after decellularization are therefore very similar. The decellularization thus demonstrably occurs very gently and can be used ideally for the method according to the invention.

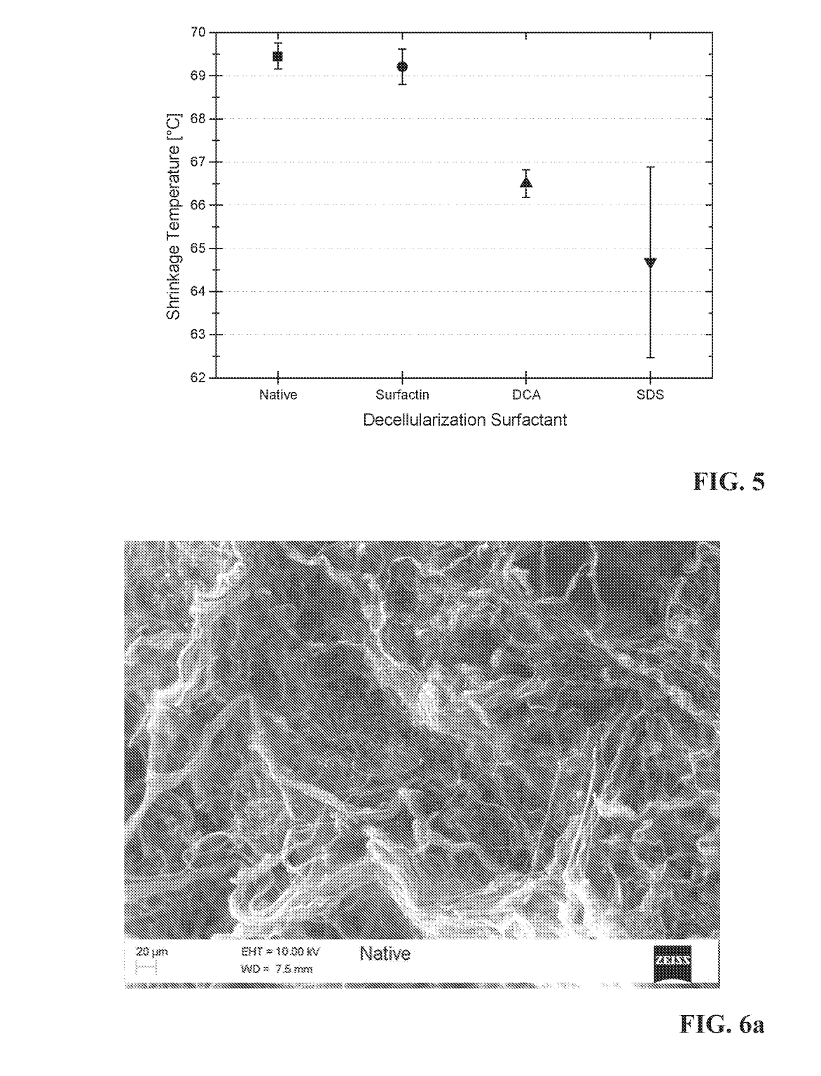

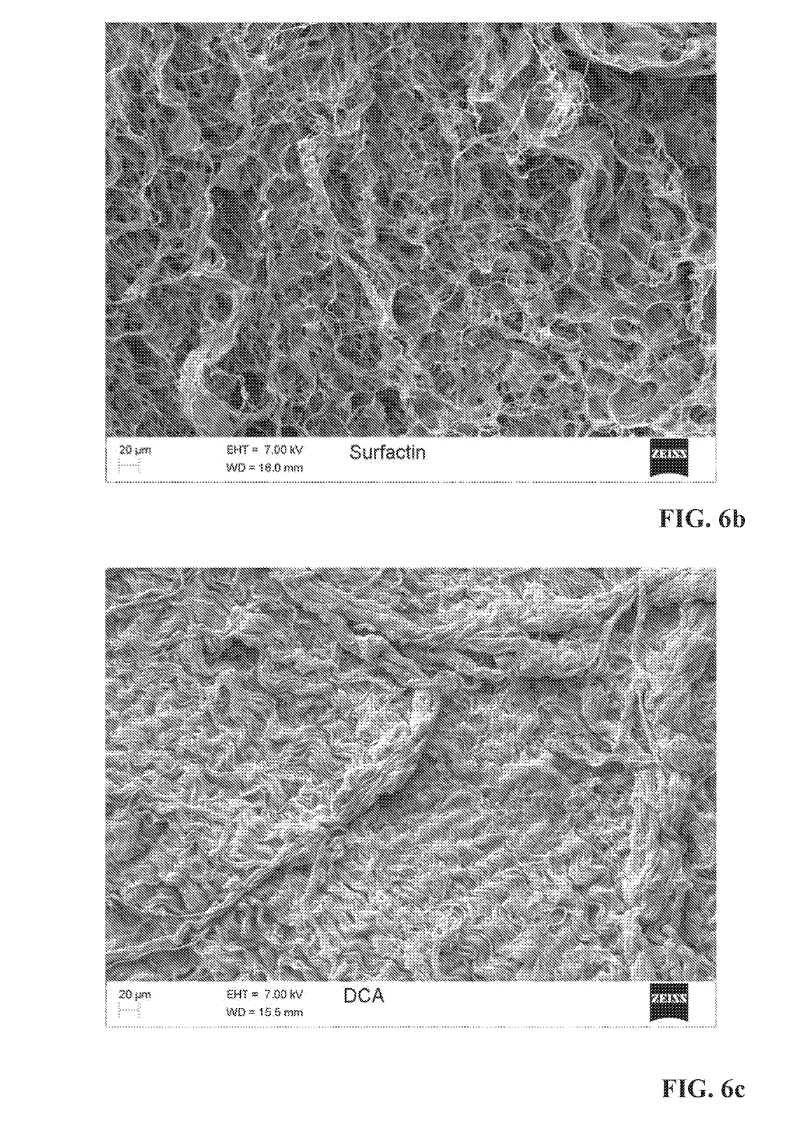

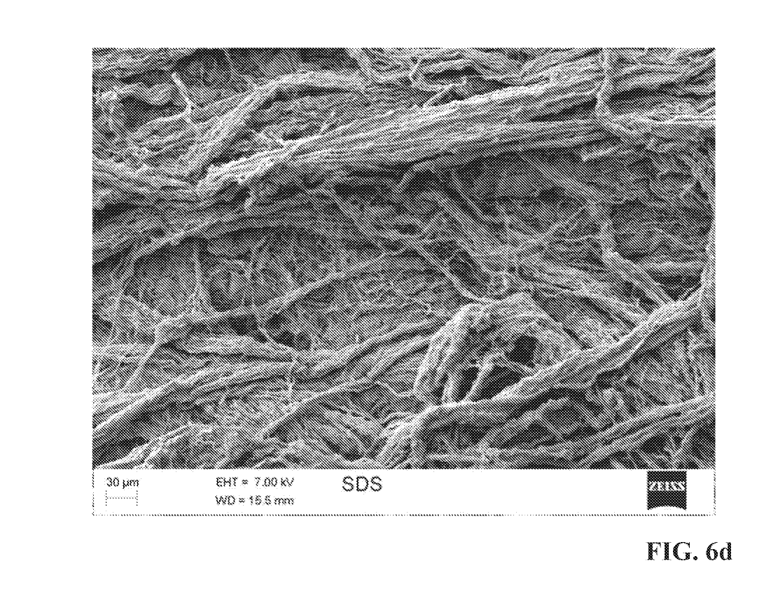

The different impairments of the tissue structure are also shown in the images, illustrated in FIGS. 6a-6d and taken by means of electron microscope, of the native tissue and of the tissue following decellularization with the detergents specified herein.

On comparison of the native tissue in FIG. 6a with the tissue in FIG. 6b decellularized with a detergent described herein, a high similarity can be seen between the recorded images. Both tissues show many collagen fibers and strands separated from one another.

By comparison, the tissue shown in FIGS. 6c and 6d following decellularization with the specified detergents according to the prior art is considerably different. In particular, smaller collagen fibers here tend to accumulate on top of one another. The tissue structure is thus changed considerably and appears to be much more compact in the images recorded by electron microscope.

### EXAMPLES

Hereinafter, an embodiment of an entire method for preparing biological tissue for implant applications according to the present proposal will be described in detail in 12 steps.

In step 1 a pericardium is removed from a pig in an abattoir and stored for 2 hours at a temperature of 4° C. in a sterile isotonic sodium chloride solution (9 g/l; company Fresenius-Kabi). The solution, besides sodium chloride, also contains penicillin and/or streptomycin in order to kill bacterial germs.

In step 2 the tissue is prepared moist in a sodium chloride solution (9 g/l; company Fresenius-Kabi). This means that here the two layers of the pericardium are separated from one another, adhering fatty and connective tissue is carefully removed, and the tissue is cut to a size and shape suitable for the desired application. Following rinsing with a sodium chloride solution (9 g/l; company Fresenius-Kabi) with slight movement of the tissue in step 3, the tissue is decellularized in step 4.

The decellularization in step 4 is performed with a detergent consisting of a surfactin-containing buffer solution. In this exemplary embodiment of the invention, surfactin (company Sigma-Aldrich, Surfactin from Bacillus subtilis, product no. F3523) with a concentration of 600 mg/l is dissolved in a DPBS phosphate buffer solution (company Lonza; DPBS w/o Ca++/Mg++; product no. 17-512). The tissue remains in this washing solution for 20 hours at 37° C. The tissue is then cleaned practically completely of cellular components located therein, without the structure of the collagen fibers being significantly changed as a result.

In step 5 the tissue is rinsed in 100 ml sodium chloride solution (9 g/l; company Fresenius-Kabi) at room temperature with slight movement. Step 5 is repeated here in this exemplary embodiment of the invention 8 times for 10 minutes.

The tissue is then treated in step 6 with α-galactosidase of Cucumis melo with a concentration of 1 unit per ml (1 U/ml) in DPBS at room temperature and a pH of 7.4 for 24 hours and is then rinsed with 200 ml DPBS. The rinsing process is repeated here six times. The α-galactosidase of Cucumis melo was commercially obtained from Sigma Aldrich.

In step 7 the tissue is rinsed for 10 minutes at 37° C. with 100 ml of a 70% ethanol solution. In step 8 a further rinsing step in 100 ml sodium chloride solution (9 g/l; company Fresenius-Kabi) is performed with slight movement.

In step 9 the collagen fibers are cross-linked with a cross-linking agent. In this exemplary embodiment of the invention the tissue is placed for 48 hours at a temperature of 4° C. in a solution containing glutaraldehyde (company Sigma-Aldrich, product no. F5882) at pH 7.4. The glutaraldehyde-containing solution consists of glutaraldehyde with a concentration of 6 g/l in DPBS without calcium and magnesium (company Lonza; DPBS w/o Ca++/Mg++; product no. 17-512).

Step 10 repeats step 9 at room temperature. Step 10 is carried out for 14 days, wherein the solution is replaced every 48 hours.

In step 11 the tissue is rinsed in this exemplary embodiment of the invention 6 times for 20 minutes at room temperature with slight movement with 100 ml sodium chloride solution (9 g/l; company Fresenius-Kabi). After a rinsing process in step 11, the tissue can be stored in glutaraldehyde or processed further in step 12.

The exemplary embodiment presented here serves to explain the invention. The number and/or embodiment of the rinsing steps (in particular concentrations, duration, temperatures and composition of the solution for rinsing the buffer solution) can be varied by a person skilled in the art within the scope of his knowledge.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

What is claimed is:

1. A method for preparing tissue for medical applications, in particular for preparing tissue for an artificial heart valve, the method comprising:
decellularizing tissue by means of a detergent,
treating the tissue with an α-galactosidase, and
cross-linking collagen fibers of the tissue by means of a suitable cross-linking agent.

2. The method according to claim 1, characterized in that the α-galactosidase is an alkaline α-galactosidase.

3. The method according to claim 1, characterized in that the α-galactosidase originates from a GH-36 family.

4. The method according to claim 1, characterized in that the α-galactosidase originates from a GH-36 family, sub-group II.

5. The method according to claim 1, characterized in that the α-galactosidase originates from Cucumis Melo.

6. The method according to claim 1, characterized in that the detergent for decellularization contains at least one lipopeptide having amphiphilic properties, consisting essentially of a hydrophilic basic structure and a hydrophobic side chain.

7. The method according to claim 1, characterized in that the detergent for decellularization contains surfactin, daptomycin, caspofungin, arthrofactin, an echinocandin, an iturin, a syringomycin, a syringopeptide and/or a polymyxin.

8. A method of using an α-galactosidase for the treatment of biological tissue for heart valve prostheses, the method comprising providing biological tissue for a heart valve prosthesis and treating the biological tissue with an α-galactosidase.

9. The method according to claim 8, characterized in that the α-galactosidase originates from the GH-36 family.

10. The method according to claim 8, characterized in that the α-galactosidase originates from Cucumis Melo.

11. A biological tissue for heart valve prostheses produced according to the method of claim 1.

12. A biological tissue for heart valve prostheses produced according to the method of claim 8.
